(19) United States
(12) Patent Application Publication
(10) Pub. No.: US 2004/0142033 A1
(43) Pub. Date: Jul. 22, 2004

(54) Pharmaceutical composition, containing oxcarbazepine with sustained release of an active-ingredient

(75) Inventors: Hanshermann Franke, Tangstedt (DE); Peter Lennartz, Hamburg (DE)

Correspondence Address: MARSHALL, GERSTEIN & BORUN LLP, 6300 SEARS TOWER, 233 S. WACKER DRIVE, CHICAGO, IL, 60606, US

(73) Assignee: DESITIN ARZNEIMITTEL GMBH, Hamburg (DE)

(21) Appl. No.: 10/723,314

(22) Filed: Nov. 26, 2003

Related U.S. Application Data
(63) Continuation-in-part of application No. PCT/EP03/05116, filed on May 15, 2003.

(30) Foreign Application Priority Data
May 31, 2002 (DE) 102 24 177.5
May 31, 2002 (DE) 102 24 170.8
Oct. 30, 2002 (DE) 102 50 566.7

(51) Int. Cl. A61K 31/55; A61K 9/22

(52) U.S. Cl. 424/468; 514/217

(57) ABSTRACT

The present invention relates to pharmaceutical compositions, in particular oral ions, with therapeutically active content of oxcarbazepine, which have a sustained of the active ingredient. The compositions have a characteristic in vitro release profile.

### CROSS-REFERENCE TO RELATED APPLICATION

This application is a continuation-in-part of application Ser. No. ______, which was forwarded on Nov. 21, 2003, which is the U.S. National Stage of international patent application PCT/03/05116, filed on May 15, 2003 and having a priority date of May 31, 2002.

### FIELD OF THE INVENTION

The present invention relates to pharmaceutical compositions, in particular compositions to be taken perorally, with an active content of oxcarbazepine.

### BACKGROUND

Oxcarbazepine is used for the treatment of epileptic diseases, for the control of neuralgic or cerebrovascular pains or for alcohol disintoxication. Oxcarbazepine is converted in the body into monohydroxydihydrocarbamazepine (MHD) which is the actual active component.

Compositions used in therapy today for peroral administration of oxcarbazepine are available exclusively in the form of peroral dosage forms with non-sustained release. After a single in viva administration, these cause the rapid increase of the plasma level of oxcarbazepine and MHD. After resorption has ended, there is then a relatively rapid decrease in the plasma concentration of the active ingredients.

The rapid active-ingredient increase of conventional compositions is associated sometimes with major side effects. In particular when administering oxcarbazepine, the occurrence of plasma peaks can lead to severe impairment of the general condition such as nausea and dizziness up to fainting. In order to avoid this, the patient must take one or more tablets two to three times a day. Only thus can a sufficiently uniform pattern of the active-ingredient level be achieved in the plasma.

However, there is an inversely proportional relationship between the degree of compliance with the prescribed drug intake during the day and the frequency of the intake per day of treatment: the more intakes per day (high intake frequency), the lower the degree of compliance, seen over the long term, with the required intake regimen (low “compliance”). Causes of this are, in addition to e.g. simply forgetting an administration, the unwillingness of patients to take medicaments in unfavourable situations. These situations typically include e.g. having meals together, business meetings or events held in groups. This applies to a particularly large degree to epilepsy patients, as today this disease still carries a social stigma.

### SUMMARY OF THE INVENTION

An object of the present invention is therefore to prepare pharmaceutical compositions for peroral administration that do not have the above-named disadvantages because, when taken once a day, they lead to a long-lasting active-ingredient level, increase at a suitable rate, of the metabolite MHD in the plasma. Minimally-active plasma levels (subtherapeutic plasma levels) must be reached. So-called plasma peaks, in particular during the initial resorption phase, should also be avoided as far as possible.

Furthermore, an object of the present invention is to provide a process for the preparation of such compounds.

The objects are achieved by a pharmaceutical composition according to claim 1 and a process according to claim 6. Surprisingly it was found that compositions, which release the following quantities of oxcarbazepine

15 mm: 55 to 85%

30 min: 75 to 95%

45 min: 85 to 100%

60 min: 90 to 100%

in vitro according to the USP paddle method (USP 24, method 724, app. 2-in 1 L 2 wt.-% sodium dodecylsulphate solution as release medium, at a stirring speed of 75 rpm), lead to a slowly increasing, long-lasting active-ingredient level of the metabolite MHD in the plasma.

On the other hand, tablets customary in the trade release the following quantities of oxcarbazepine according to the same release method (see FIG. 3)

15 min: approx. 88 to 90%

30 mm: approx. 95 to 100%

45 min: approx. 98 to 100%

60 min: approx. 100%

and have the above-named disadvantages.

The result is surprising because the in vitro release pattern of oxcarbazepine of the compositions according to the invention is only slightly below that of tablets commonly marketed, at which a sufficient prolongation of the action is usually not expected. On the other hand, typical sustained release formulations with a subsequently low in vitro release profile (60 min: approx. 40% oxcarbazepine release) have proved ineffective.

### BRIEF DESCRIPTION OF THE DRAWINGS

FIG. 1 is a microscopic photo (80×) of a preferred particle size distribution of the compacted product;

FIG. 2 is a diagram of a DSC test on oxcarbazepine active;

FIG. 3 is a diagram of a DSC test on the compacted product;

FIG. 4 is a graph showing the oxcarbazepine release pattern for the tablet of Example 2;

FIG. 5 is a graph showing the plasma level pattern of oxcarbazepine after peroral administration of tablets, prepared according to Example 2, containing 600 mg oxcarbazepine;

FIG. 6 is a graph showing the oxcarbazepine release patter for a comparison tablet; and

FIG. 7 is a graph showing the plasma level patter of oxcarbazepine after peroral administration of tablets customary in the trade (TRILEPTOL of Novartis) containing 600 mg of oxcarbazepine.

### DETAILED DESCRIPTION

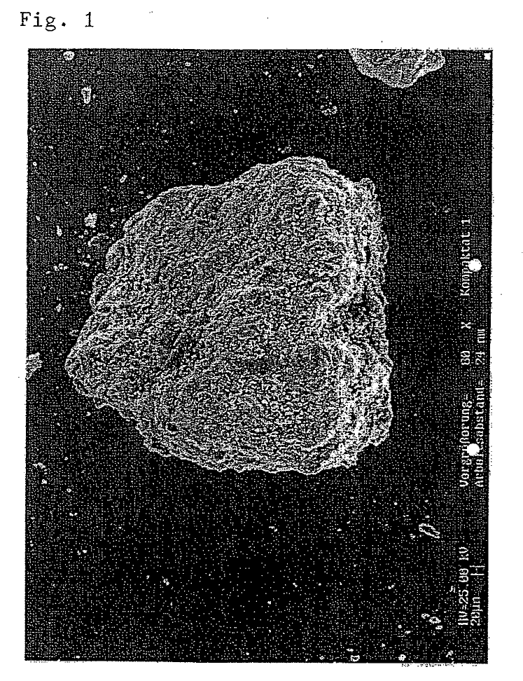

Preferred embodiments according to the invention are described below, where FIG. 1 shows a preferred particle size distribution of the compacted product according to the invention.

FIG. 2 shows the diagram of a DSC test on oxcarbazepine active.

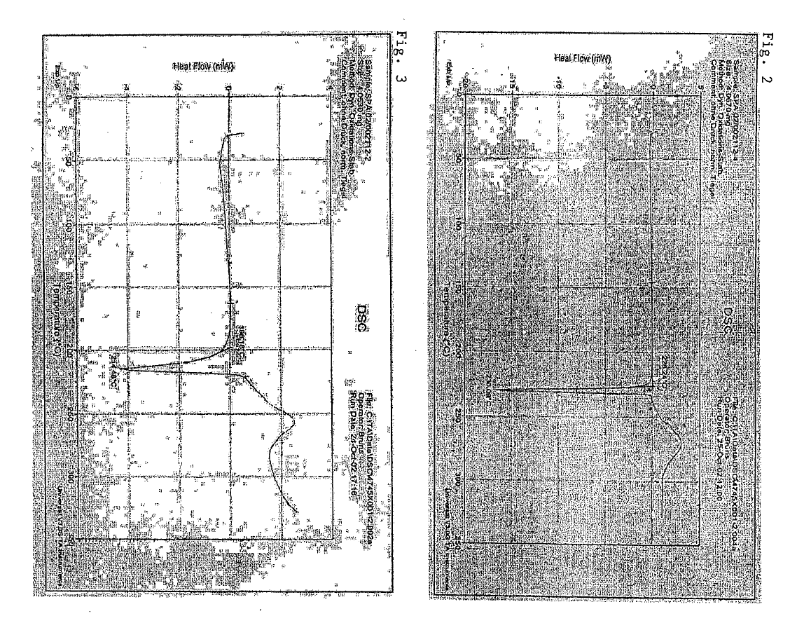

FIG. 3 shows the diagram of a DSC test on the compacted product according to the invention.

The compositions according to the invention preferably release the following quantities of oxcarbazepine:

15 min: 65 to 80%

30 min: 85 to 95%

45 min: 90 to 100%

60 mm: 95 to 100%

in vitro according to the USP paddle method (USP 24, method 724, app. 2 in 1 L 2 wt.-% sodium dodecylsulphate solution as release medium, at a stirring speed of 75 rpm)

After peroral intake of the composition according to the invention, containing 600 mg of oxcarbazepine, the following plasma concentrations of oxcarbazepine are preferably achieved:

| | |
|---|---|
| 1.5 to 2 hours | 0.2 to 0.6 mg/L |
| 5.5 to 6.5 hours | 0.1 to 0.3 mg/L |
| 11 to 13 hours | 0.1 to 0.2 mg/L |
| 23 to 25 hours | 0.0 to 0.2 mg/L |

and the following plasma concentrations of MHD:

| | |
|---|---|
| 1.5 to 2 hours | 1 to 4 mg/L |
| 5.5 to 6.5 hours | 3 to 5 mg/L |
| 11 to 13 hours | 3 to 5 mg/L |
| 23 to 25 hours | 1.5 to 4.5 mg/L |

After peroral intake of the composition according to the invention, containing 600 mg oxcarbazepine, the following plasma concentrations of oxcarbazepine are particularly preferably achieved:

| | |
|---|---|
| 1.5 to 2 hours | 0.3 to 0.5 mg/L |
| 5.5 to 6.5 hours | 0.1 to 0.4 mg/L |
| 11 to 13 hours | 0.1 to 0.2 mg/L |
| 23 to 25 hours | 0.0 to 0.1 mg/L |

and the following plasma concentrations of

| | |
|---|---|
| 1.5 to 2 hours | 1 to 3 mg/L |
| 5.5 to 6.5 hours | 3.5 to 4.5 mg/L |
| 11 to 13 hours | 3.5 to 4.5 mg/L |
| 23 to 25 hours | 2.5 to 4 mg/L. |

The pharmaceutical composition according to preferably produces an average plasma level to 5 mg/mL and a maximum plasma level (Cₘₐₓ) to 5 of MHD of 3 mg/mL in vivo after peroral intake of the composition, containing 600 mg oxcarbazepine, in the period from 4 hours after intake to 21 hours after intake.

The compositions according to the invention can be prepared by preparing and then compacting a mixture which, relative to its total weight, contains

A. 60 to 95 wt.-% oxcarbazepine,

B. 3 to 30 wt.-% microcrystalline cellulose,

C. 1 to 20 wt.-% ammonium methacrylate copolymer and/or polymethacrylic acid polymer,

D. 0.05 to 4 wt.-% disintegrant and

E. dye.

The mixture preferably contains, relative to its total weight:

A. 80 to 90 wt.-% oxcarbazepine,

B. 5 to 15 wt.-% microcrystalline cellulose,

C. 2 to 10 wt.-% ammonium methacrylate copolymer and/or polymethacrylic acid polymer,

D. 0.1 to 2 wt.-% disintegrant and

E. dye.

Suitable disintegrants are in particular sodium carboxymethyl starch, croscarmellose sodium and polyvinylpolypyrrolidone.

In the case of oxcarbazepine preparations, the use of dyes is customary due to the possible formation of coloured decomposition products. None of the iron oxides/iron hydroxides frequently used as dye are used in the composition according to the invention, as these can favour the formation of decomposition products from oxcarbazepine. The resulting iron intake in the case of high-dose drugs such as oxcarbazepine can also be toxicologically unacceptable. Organic compounds and lacquer from organic compounds can be used as dyes. Riboflavin and yellow-orange S lacquer in particular are suitable.

The thus-obtained compacted material has very good flow properties and therefore requires no further addition of a flow-regulating means such as colloidal silicic acid (e.g. Aerosil 200®). In particular colloidal silicic acid can cause the formation of undesired decomposition products from oxcarbazepine.

The thus-obtained compacted material can then be classified and packed into hard gelatin capsules or packed into small pouches (sachets). However, tablets are preferably prepared from the compacted material by initially adding to same, relative to 100 parts by weight of the compacted material,

F. 0.2 to 5 parts by weight tablet lubricant and

G. 10 to 50 parts by weight microcrystalline cellulose

and further processing the thus-obtained mixture into a tablet.

In particular magnesium stearate and calcium stearate can be used as tablet lubricant.

The thus-obtained tablets can be coated with a film in a drum coater, using water and, relative to 100 parts by weight of the compacted material,

F. 0.5 to 10 parts by weight polymethacrylic acid copolymer

G. 0.025 to 2 parts by weight plasticizer

H. 0.025 to 2 parts by weight anti-adherent agent

I. dyes and pigments q.s.

The thus-obtained tablets can also be coated with a film in a drum coater, using water and, relative to 100 parts by weight of the compacted material,

F. 0.5 to 10 parts by weight film former

G. 0.0 to 2 parts by weight plasticizer

H. 0.005 to 2 parts by weight anti-adherent agent

I. dyes and pigments q.s.

In particular cellulose derivatives or polyacrylic acid derivatives can be used as film formers.

In particular triethyl citrate, triacetin can be used as plasticizers.

In particular talcum, glyceryl monostearate can be used as anti-adherent agents.

The compacted material can also be coated with a film in the fluidized bed or in the high-shear mixer with the addition of water, using, relative to 100 parts by weight of the compacted material,

F. 0.5 to 10 parts by weight polymethacrylic acid copolymer

G. 0.025 to 2 parts by weight plasticizer

H. 0.025 to 2 parts by weight anti-adherent agent

The compounds named above in each case can be used as plasticizers and anti-adherent agents.

A granulated material is obtained which can then be classified and packed into hard gelatin capsules or packed into small pouches (sachets). However, tablets are preferably prepared from the granulated material by initially adding to same, relative to 100 parts by weight of the granulated material,

I. 0.2 to 0.5 parts by weight tablet lubricant and

J. 10 to 50 parts by weight microcrystalline cellulose

and further processing the thus-obtained mixture into a tablet.

In particular magnesium stearate and calcium stearate can be used as tablet lubricant.

The compositions according to the invention can also be prepared by preparing a granulated material which, relative to its total weight, contains

A. 60 to 95 wt.-% oxcarbazepine

B. 3 to 30 wt.-% microcrystalline cellulose

C. 0.05 to 4 wt.-% disintegrant

D. 1 to 20 wt.-% polymer

E. 0.2 to 5 wt.-% plasticizer

F. 0 to 5 wt.-% anti-adherent agent

G. dye

in the fluidized bed or in the high-shear mixer, with the addition of water. The granulated material preferably contains, relative to its total weight:

A. 80 to 90 wt.-% oxcarbazepine

B. 5 to 15 wt.-% microcrystalline cellulose

C. 0.1 to 2 wt.-% disintegrant

D. 2 to 10 wt.-% polymer

E. 0.4 to 2.5 wt.-% plasticizer

F. 0 to 0.25 wt.-% anti-adherent agent

G. dye.

In particular polymethacrylic acid ester, ammonium methacrylate copolymer can be used as polymers.

The compounds named above in relation to the preparation of tablets are preferably used as plasticizers and anti-adherent agents.

In particular the following substances can be used as disintegrants: sodium carboxymethyl starch, croscarmellose sodium and polyvinylpolypyrrolidone.

In particular organic dyes and organic lacquers can be used as dyes.

The thus-obtained granulated material can then be packed into hard gelatin capsules or packaged into small pouches (sachets). However, tablets are preferably prepared from the granulated material by initially adding to same, relative to 100 parts by weight of the granulated material,

H. 0.2 to 0.5 parts by weight tablet lubricant and

I. 10 to 50 parts by weight microcrystalline cellulose

and further processing the thus-obtained mixture into a tablet.

In particular magnesium stearate and calcium stearate can again be used as tablet lubricant.

An economical process with high production output rates is established by using the starting material oxcarbazepine with an optimum particle size distribution. While to a certain degree fine particles contribute to inefficient process parameters, unfavourable amounts of coarse particles cause non-cohesiveness and therefore mechanically unstable tablets.

A favourable particle size of the oxcarbazepine lies within the following range:

Determination by laser beam diffraction (Malvern Mastersizer, dry dispersion)

d (0.1)=20 μm-70 μm

d (0.5)=70 μm-175 μm

d (0.9)=200 μm-450 μm

Preferably, the particle size of the oxcarbazepine lies within the following range:

d (0.1)=25 μm-45 μm

d (0.5)=90 μm-125 μm

d (0.9)=250 μm-350 μm

The compacted product according to the invention preferably exhibits the following particle size distribution:

Determination by sieve analysis (Retsch AS control)

>1.000 mm=0%-5%

1.000 mm-0.500 mm=35%-65%

0.500 mm-0.250 mm=15%-35%

0.250 mm-0.125 mm=10%-25%

0.125 mm-0.063 mm=0%-15%

<0.063 mm-0%-5%

Compacted products according to the invention exhibiting the above described particle size show positive production characteristics like good flowability and mechanical strength of the resulting dosage forms.

The pharmaceutical compositions according to the invention can advantageously be used for the preparation of a drug for the prevention or the treatment of primarily generalized tonic-clonic seizures and/or focal seizures with or without secondary generalization.

### EXAMPLES

### Example 1

### Preparation of Compacted Material

Preparation of compacted material 30 kg oxcarbazepine were mixed for 5 minutes in a high-shear mixer with 2 kg ammonium methacrylate copolymer (Eudragit RSPO®), 4 kg microcrystalline cellulose and 0.4 kg sodium carboxymethyl starch. The resultant mixture was compacted in a compactor (3-W-Polygrane of the company Gerteis Maschinen+Processengineering A G, Jona, Switzerland). The resultant ribbons were crushed by means of forced screening and the resultant compacted material is classified via a vibrating screen (1 mm screen tray, vibrating screen of the company Engelsmann, screen channel with 0.25 mm screen tray).

A part of the classified compacted material was packed into hard gelatin capsules of sizes 3, 2, 1 and 0 on a capsule-packing machine. Doses of 150 to 300 mg oxcarbazepine per single dose resulted.

A further part of the classified compacted material was packed into small pouches (sachets) on a bagging machine. Doses of 50 to 2400 mg oxcarbazepine per single dose resulted.

### Example 2

### Preparation of Tablets

A compacted material was prepared and classified according to Example 1. The compacted material was mixed with 0.5 kg magnesium stearate and 8 kg microcrystalline cellulose and then pressed into tablets, wherein doses between 150 and 600 mg oxcarbazepine resulted per tablet.

### Example 3

### Examination of the In Vitro Release Pattern

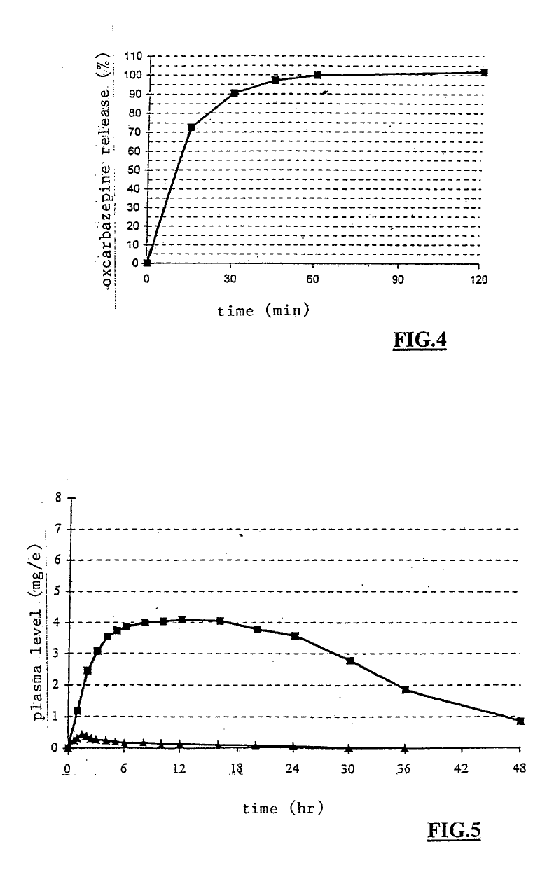

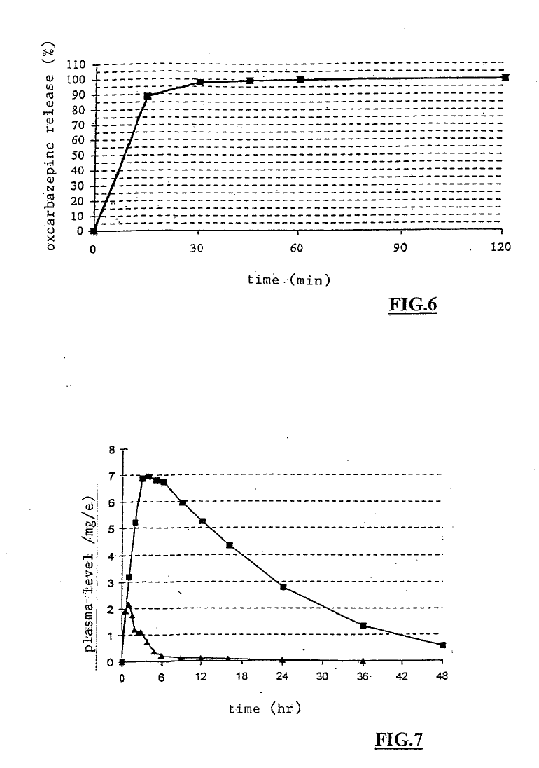

A tablet prepared according to Example 2 containing 600 mg oxcarbazepine was examined in vitro according to the USP paddle method (USP 24, method 724, app. 2 in 1 L 2 wt.-% sodium dodecylsulphate solution as release medium, at a stirring speed of 75 rpm) and the release pattern compared with the tablet that is customary in the trade (Trileptal of the company Novartis). The release pattern of the tablet according to the invention is reproduced in FIG. 4 and the release pattern of the comparison tablet is reproduced in FIG. 6. It is shown that the release of oxcarbazepine in vitro proceeds only slightly more slowly. CL Example 4

### Examination of the Plasma Level

Tablets prepared according to Example 2 containing 600 mg oxcarbazepine were administered to subjects and the plasma level pattern of oxcarbazepine and MHD was recorded. The results of the tests (arithmetic means) are reproduced in FIG. 5. Therein the closed triangles indicate the values for oxcarbazepine and the closed squares indicate the values for MHD.

As a comparison, tablets customary in the trade containing 600 mg oxcarbazepine (Trileptal of the company Novartis) were administered and the plasma level pattern of oxcarbazepine and MHD was recorded. The results of the tests (arithmetic means) are reproduced in FIG. 7. Therein the filled triangles indicate the values for oxcarbazepine and the filled squares indicate the values for MHD.

The figures show that the MHD plasma level of the compositions according to the invention rises slowly to a maximum concentration of roughly 3 to 5 mg/L and remains roughly constant over a period from roughly 4 hours after intake to 24 hours after intake. On the other hand, the MHD plasma level of the comparison compositions rises rapidly to a value of roughly 7 mg/L and then falls rapidly again.

1. Pharmaceutical composition, containing oxcarbazepine, which releases the following quantities of oxcarbazepine:
15 min: 55 to 85%
30 min: 75 to 95%
45 min: 85 to 100%
60 min: 90 to 100%
in vitro according to the USP paddle method (USP 24, method 724, app. 2 in 1 L 2 wt.-% sodium dodecylsulphate solution as release medium, at a stirring speed of 75 rpm).

2. Pharmaceutical composition according to claim 1, containing oxcarbazepine, which releases the following quantities of oxcarbazepine:
15 min: 65 to 80%
30 mm: 85 to 95%
45 min: 90 to 100%
60 min: 95 to 100%
in vitro according to the USP paddle method (USP 24, method 724, app. 2 in 1 L 2 wt.-% sodium dodecylsulphate solution as release medium, at a stirring speed of 75 rpm).

3. Pharmaceutical composition according to claim 1, which produces the following plasma concentrations of oxcarbazepine:

| | |
|---|---|
| 1.5 to 2 hours | 0.2 to 0.6 mg/L |
| 5.5 to 6.5 hours | 0.1 to 0.3 mg/L |
| 11 to 13 hours | 0.1 to 0.2 mg/L |
| 23 to 25 hours | 0.0 to 0.2 mg/L |

in vivo after peroral intake of the pharmaceutical composition, in such a way that 600 mg oxcarbazepine are administered, and which produces the following plasma concentrations of monohydroxydihydrocarbamazepine:

| | |
|---|---|
| 1.5 to 2 hours | 1 to 4 mg/L |
| 5.5 to 6.5 hours | 3 to S mg/L |
| 11 to 13 hours | 3 to 5 mg/L |
| 23 to 25 hours | 2.5 to 4.5 mg/L. |

4. Pharmaceutical composition according to claim 1, which, in vivo after peroral intake of the pharmaceutical composition, in such a way that 600 mg oxcarbazepine are administered, produces an average plasma level of monohydroxydihydrocarbamazepine of 3 to 5 mg/mL in the period from 4 hours after intake to 21 hours after intake.

5. Pharmaceutical composition according to claim 1, which, in vivo after peroral intake of the pharmaceutical composition, in such a way that 600 mg oxcarbazepine are administered, produces a maximum plasma level (Cₘₐₓ) of monohydroxydihydrocarbamazepine of 3 to S mg/mL.

6. Process for the preparation of a pharmaceutical composition according to claim 1, in which a mixture which, relative to its total weight, contains
a. 60 to 95 wt.-% oxcarbazepine,
b. 3 to 30 wt.-% microcrystalline cellulose,
c. 1 to 20 wt.-% ammonium methacrylate copolymer and/or polymethacrylic acid polymer,
d. 0.05 to 4 wt.-% disintegrant and
e. dye
is prepared and then compacted.

7. Process according to claim 6, in which a mixture which, relative to its total weight, contains
a. 80 to 90 wt.-% oxcarbazepine,
b. 5 to 15 wt.-% microcrystalline cellulose,
c. 2 to 10 wt.-% ammonium methacrylate copolymer and/or polymethacrylic acid polymer,
d. 0.1 to 2 wt.-% disintegrant and
e. dye
is prepared and then compacted.

8. Process according to claim 6, in which the compacted material is screened and packed into capsules or into pouches unchanged or optionally provided with excipients.

9. Process according to claim 6, in which after the compacting, relative to 100 parts by weight of the compacted material,
f. 0.2 to 5 parts by weight magnesium stearate and
g. 10 to 50 parts by weight microcrystalline cellulose
are added and the thus-obtained mixture is further processed into a tablet.

10. Process for the preparation of a pharmaceutical composition according to claim 1, in which a granulated material which, relative to its total weight, contains
A. 60 to 95 wt.-% oxcarbazepine
B. 3 to 30 wt.-% microcrystalline cellulose
C. 0.05 to 4 wt.-% disintegrant
D. 1 to 20 wt.-% polymer
E. 0.2 to 5 wt.-% plasticizer
F. 0 to 5wt. -% anti-adherent agent
G. dye
is prepared in a fluidized bed or in a high-shear mixer with the addition of water.

11. Process according to claim 10, in which the granulated material, relative to its total weight, contains:
A. 80 to 90 wt.-% oxcarbazepine
B. 5 to 15 wt.-% microcrystalline cellulose
C. 0.1 to 2 wt.-% disintegrant
D. 2 to 10 wt.-% polymer
E. 0.4 to 2.5 wt.-% plasticizer
F. 0 to 2.5 wt.-% anti-adherent agent
G. dye q.s.

12. Process according to claim 10, in which, relative to 100 parts by weight of the granulated material,
H. 0.2 to 0.5 parts by weight tablet lubricant and
I. 10 to 50 parts by weight microcrystalline cellulose
are added and the thus-obtained mixture is further processed into a tablet.

13. Process according to claim 6, in which the compacted material, using relative to 100 parts by weight of the compacted material,
F. 0.5 to 10 parts by weight polymethacrylic acid copolymer
G. 0.025 to 2 parts by weight plasticizer
H. 0.025 to 2 parts by weight anti-adherent agent
is coated with a film in the high-shear mixer with the addition of water.

14. Process according to claim 13, in which, relative to 100 parts by weight of the film-coated compacted material,
I. 0.2 to 0.5 parts by weight tablet lubricant and
J. 10 to 50 parts by weight microcrystalline cellulose
are added and the thus-obtained mixture is further processed into a tablet.

15. Process according to claim 9, in which the tablets are coated with a film in a drum coater, using water and, relative to 100 parts by weight of the tablet,
H. 0.5 to 10 parts by weight polymethacrylic acid copolymer
I. 0.025 to 2 parts by weight plasticizer
J. 0.025 to 2 parts by weight anti-adherent agent, and
K. dye and/or pigments.

16. Process according to claim 9, in which the tablets are coated with a film in a drum coater, using water and, relative to 100 parts by weight of the tablets,
H. 0.5 to 10 parts by weight film former
I. 0.0 to 2 parts by weight plasticizer
J. 0.005 to 2 parts by weight anti-adherent agent, and
K. dye and/or pigments.

17. Pharmaceutical composition which is obtained according to the process of to claim 7.

18. A process for the treatment of primarily generalized tonic-clonic seizures and/or focal seizures, with or without secondary generalization, comprising perorally administering an effective amount of the pharmaceutical composition according to claim 1.

19. A process for the treatment of neuralgic and cerebrovascular pains or for alcohol disintoxication, comprising perorally administering an effective amount of the pharmaceutical composition according to claim 1.

20. Pharmaceutical composition according to claim 1, which contains oxcarbazepine of the following particle size distribution:
Determination by laser beam diffraction (Malvern Mastersizer, dry dispersion)
d(0.1)=20 μm-70 μm
d(0.5)=70 μm-175 μm
d(0.9)=200 μm-450 μm

21. Pharmaceutical composition according to claim 1, which contains oxcarbazepine of the following particle size distribution:
Determination by laser beam diffraction (Malvern Mastersizer, dry dispersion)
d(0.1)=25 μm-45 μm
d(0.5)=90 μm-125 μm
d(0.9)=250 μm-350 μm

22. Process according to claim 8, in which the particle size of the compacted product lies within the following range:
Determination by sieve analysis (Retsch AS control)
>1.000 mm=0%-5%
1.000 mm-0.500 mm=35%-65%
0.500 mm-0.250 mm=15%-35%
0.250 mm-0.125 mm=10%-25%
0.125 mm-0.063 mm=0%-15%
<0.063 mm-0%-5%
